(19) United States
(12) Patent Application Publication
(10) Pub. No.: US 2010/0144668 A1
(43) Pub. Date: Jun. 10, 2010

(54) SEMI-FLUID FOOD PRODUCT INCLUDING BETA GLUCANE FIBRES AND GUAR GUM, AND USE THEREOF AS A FUNCTIONAL FOOD PRODUCT

(76) Inventors: Sophie Vinoy, Athis Mons (FR); Tatiana Steiler, Lamorlaye (FR); Pascale Rondeau, Savigny Sur Orge (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/527,501

(22) PCT Filed: Feb. 19, 2008
(86) PCT No.: PCT/EP08/52009

§ 371 Date: Jan. 20, 2010

(30) Foreign Application Priority Data
Feb. 20, 2007 (FR) 0753384

(51) Int. Cl. A61K 31/716; A23L 1/0526; A23L 1/06; A23L 1/29; A23L 1/308; A61P 3/10; A61P 3/04

(52) U.S. Cl. 514/54; 426/629; 426/580; 426/634; 426/623; 426/2

(57) ABSTRACT

The invention relates to a semi-fluid food product containing from 2.5 to 16 g of guar gum and from 2.8 to 11.3 g of beta-glucane fibres per portion of said food product, said portions ranging from 125 to 250 g, characterised in that the guar gum: beta-glucane fibres mass ratio ranges from 2:1 to 4:1.

The present invention relates to novel food products including dietary fibres (guar gum and beta-glucan fibres), intended to decrease the insulinaemia response following consumption of a meal.

The ingestion of a typical meal, supplying proteins, lipids and carbohydrates, is quickly followed by an increase in glycaemia due to the absorption of carbohydrates.

Glucose and fatty acids (FAs) are the body's two principal sources of energy and their uses are interconnected. Their respective degree of use is determined by insulin. In the absence of insulin, i.e., long after a meal, glucose is used only very slightly by insulin-dependent tissues (muscles, adipose tissue) and lipolysis and circulating FAs are high. The intensity of the oxidation of FAs is determined by their concentration in the blood, i.e., it follows the law of mass action (Zurlo F., Lillioja S., Esposito-Del Puente A., Nyomba B. L., Raz I., Saad M. F., Swinburn B. A., Lissner L., Heitmann B. L. Dietary fat and obesity: evidence from epidemiology. Eur. J. Clin. Nutr., 1995; 49: 79-90). That of glucose would follow the same law in the absence of insulin. The balance between carbohydrate oxidation and lipid oxidation is thus achieved on the basis of simple competition for substrates, taking into account that the use of glucose is given priority by insulin which induces its secretion (Kelley D. E., Mokan M., Simoneau J. A., Mandarino L. J. Interaction between glucose and free fatty acid metabolism in human skeletal muscle. The Journal of Clinical Investigation 1993; 92: 91-8).

The situation immediately postprandial (the first two hours after a meal) is a combination of hyperglycaemia and the hyperinsulinaemia it causes. A consequence of this hormonal change is stimulation of the use of glucose by insulin-sensitive tissues, principally by an increase in glucose transport in cells. In addition, any rise in the concentration of insulin in the blood corresponds to a drop in the concentration of circulating FAs. Indeed, insulin inhibits the mobilisation of stored lipids and favours their storage in fat tissues (Sadur C. N., Eckel R. H. Insulin stimulation of adipose tissue lipoprotein lipase. Use of the euglycemic clamp technique. The Journal of Clinical Investigation 1982; 69:1 119-25; Strålfors P., Björgell P., Belfrage P. Hormonal regulation of hormone-sensitive lipase in intact adipocytes: identification of phosphorylated sites and effects on the phosphorylation by lipolytic hormones and insulin. Proceedings of the National Academy of Sciences of the United States of America 1984; 3317-21).

Thus, just after a meal, carbohydrate oxidation and the storing of FAs in reserve in the form of triglycerides (TGs) in adipose tissue are all the more intense since insulin secretion is high. This depends on the quantity and quality of simple carbohydrates (for example fructose and glucose) as well as the speed of absorption of ingested carbohydrates (so-called “slow” or “fast” carbohydrates). Then glycaemia and insulinaemia will gradually drop—lipid synthesis initially—and then the impediment of mobilisation of stored lipids will ease little by little. To these endogenous FAs will be added FAs absorbed during the meal and the oxidation of these two sources will then save glucose, more or less delaying the hypoglycaemia “hunger signal”. FAs still circulating after the release of the meal will be stored owing to the hyperglycaemia episode which will follow and may be mobilised later when insulinaemia allows it once again (Gómez F., Jéquier E., Chabot V., Büber V., Felber J. P. Carbohydrate and lipid oxidation in normal human subjects: its influence on glucose tolerance and insulin response to glucose. Metabolism: clinical and experimental 1972; 21: 381-91).

Current consumption habits supply a higher quantity of energy than the quantity of energy expended daily. This energy surplus is stored by the body via higher insulin secretion.

How to remediate this situation? First, the consumption of energy beyond daily energy requirements must be limited. One means is to decrease the fat content of food, as well as to reduce insulin secretion by modifying the supply of carbohydrates in such a way that they cause less insulin secretion. Thus, these two levers enable an improvement in the quantity of energy absorbed but also its destiny in the body via insulin.

The inventors thus had the aim of identifying ingredients likely to decrease the insulinaemia response of a meal in subjects, in particular healthy subjects of normal weight or overweight subjects (20<BMI<30) while maintaining a relatively low glycaemic response.

Numerous studies on the impact of soluble dietary fibres were carried out. Studies of note include those of Spiller et al., which describe the hypolipidaemia effect of fibres of guar gum or of β-glucan (Guar gum and plasma cholesterol. Effect of guar gum and an oat fibre source on plasma lipoproteins and cholesterol in hypercholesterolemic adults. G. A. Spiller, J. W. Farquhar, J. E. Gates and S. F. Nichols Arterioscler. Thromb. Vasc. Biol. 1991; 11; 120-), the study of Begin et al., which describes the effect of soluble dietary fibres (guar gum, carboxymethylcellulose, mustard mucilage, or oats β-glucan) on glycaemia and insulinaemia (Effect of dietary fibres on glycaemia and insulinemia and on gastrointestinal function in rats. Begin F., Vachon C., Jones J. D., Wood P. J., Savoie L., Can J Physiol Pharmacol. October 1989; 67 (10): 1265-71) or the study of Vachon et al., which describes that soluble dietary fibres (carboxymethylcellulose, guar gum, oat β-glucan or mustard mucilage) have a positive effect on postprandial insulinaemia but little effect on glycaemia (Concentration effect of soluble dietary fibers on postprandial glucose and insulin in the rat. Vachon C., Jones J. D., Wood P. J., Savoie L., Can J Physiol Pharmacol. 1988 June; 66 (6): 801-6). The American application US 2004/0096479 described a dietary supplement very rich in fibres, these fibres consisting of at least a mixture of three fibres: guar gum, oats and psyllium.

After ingestion of a meal, glycaemic and insulinaemic responses can be measured and monitored over time by taking regular blood samples (cf. FIG. 1).

After ingestion of a meal, the glycaemia profile over time shows a peak followed by a return to the basal value after two hours. The insulinaemia profile follows that of the glycaemia profile. Peak and area under the curve (AUC) are two indicators of postprandial glycaemic and insulinaemic profiles.

The objective of the inventors is to identify novel food products that decrease the peak and area under the curve of insulinaemia, while maintaining a normal glycaemic profile.

The inventors discovered that a combination of guar gum and β-glucan fibres can decrease, with a synergistic effect, the insulinaemic response while maintaining a normal glycaemic profile.

Thus, the first aim of the invention is a food product including 2.5 g to 16 g of guar gum and 2.8 g to 11.3 g of β-glucan fibres, per portion of said food product. In the inventive food product the weight ratio of guar gum to β-glucan fibre is between 2:1 and 4:1, in an advantageous way said ratio is 2:1.

The inventive food product advantageously includes 8 g to 16 g of guar gum, more advantageously 8 g to 12 g of guar gum, even more advantageously 8 g of guar gum, and 3 g to 4 g of β-glucan fibres, advantageously 3 g of β-glucan fibres, per portion of said food product.

In the context of the invention, the portions of the food product are between 125 g and 250 g, advantageously between 150 g and 250 g, more advantageously they are 150 g.

Guar gum comes from the endosperm of seeds of guar, Cyamopsis tetragonolobus, also called Indian cluster bean.

It is composed of approximately 83% high molecular weight galactomannans, a water soluble fibre that is highly viscous, which makes it a good thickener or stabiliser for food products. It is not consumed as such in current diets.

Guar gum can be partially hydrolysed by an enzymatic process, which reduces its viscosity and influences its properties. The degree of viscosity is proportional to the level of galactomannans in the molecule.

In the context of the present invention, the guar gum is advantageously partially hydrolysed guar gum. The molecular weight of guar gum advantageously is between 10 kDa and 300 kDa, more advantageously between 10 kDa and 100 kDa, even more advantageously between 10 kDa and 30 kDa, in particular the molecular weight of guar gum is 20 kDa.

Guar gum is dispersed in the inventive food product; it can for example be introduced in the form of a syrup.

β-glucans are polysaccharides extracted from the cell wall of green plants, cereals (oats and barley) and certain algae and mushrooms (maitake and shiitake). They are primarily composed of glucose molecules or their derivative, bound together by β bonds (β1-3 or β1-4 and/or β1-6).

It is a soluble and viscous fibre whose viscosity depends on molecular weight and amount provided.

The source of β-glucan fibres is generally an extract of oats or barley, enriched in said fibres. However, it should be noted that oats or barley flours or brans have a very low content of β-glucan fibres (approximately 3% by weight (w/w=weight/weight) only in oat bran, from 0.1% to 4% by weight (w/w) in oat flour), which rends their use as a source of β-glucan fibres difficult to envisage. On the other hand, purified extracts of β-glucan fibres are available commercially. In particular, Cargill sells a purified extract of barley β-glucan with a β-glucan content higher than 70% (under the trade name Barliv™).

Oat β-glucan fibres, whose molecular weight is higher, are preferred to barley β-glucan fibres. In addition, although all forms of barley seeds can be used, advantageously complete amylose-rich barley seeds are used (one such extract is sold by Cargill under the trade name Barliv™).

The inventive food product is semi-fluid and can contain solid ingredients such as crisps rich in oat β-glucan fibres.

In the context of the present invention, a semi-fluid food product has a water activity (aw) higher than 0.90 (water activity is the ratio of the vapour pressure of a product to the vapour pressure of pure water at the same temperature).

Guar gum is introduced into the product in the form of a water-based syrup and guar gum powder or in the form of fruit preparations containing water, fruits, sugar, stabilisers and guar gum powder.

β-glucan fibres can be either dispersed in the inventive food product or introduced into said food product in a solid form:
β-glucan fibres (in particular oat extracts very low in β-glucan) can be introduced via crisps contained in a separate compartment, for example placed above the yogurt container, and mixed in the semi-fluid product at the last moment;
β-glucan fibres (in particular extracts from barley and enriched in β-glucan such as Barliv™) can be introduced via a patented process whose description is given below.

According to a first variant of the invention, β-glucan fibres are dispersed in said food product.

In the context of this first variant β-glucan fibres are introduced into the food product according to the following method:
a) preparation of a semi-fluid thermized aqueous solution including β-glucan fibres and at least partially hydrolysed guar gum;
b) introduction of this thermized solution into said food product.

“Thermized” means treated with heat to eliminate microbiological contaminants. This treatment can be pasteurisation, sterilisation, or any other thermal process.

“Semi-fluid” means a solution that has a viscosity (measured at 10° C.) lower than 10,000 mPa s.

In addition to a strong texturing capacity, β-glucan fibres also have a strong gelling capacity. However, the inventors showed that it was possible to prepare a semi-fluid thermized aqueous solution containing a significant quantity of β-glucan fibres by the use of at least partially hydrolysed guar gum combined with the use of a slow cooling process under shearing.

In the context of this variant, the at least partially hydrolysed guar gum advantageously has a molecular weight between 10 kDa and 100 kDa, more advantageously between 10 kDa and 50 kDa, even more advantageously between 10 kDa and 30 kDa. In particular, the at least partially hydrolysed guar gum can have a molecular weight of approximately 20 kDa. This at least partially hydrolysed guar gum is advantageously obtained following enzymatic hydrolysis of the guar gum.

The minimal content of the at least partially hydrolysed gum guar as a viscosity reducer in the inventive aqueous solution varies from 5% to 30% by weight, compared to the total weight of said solution.

The content of viscosity reducer depends on the one hand on the content of β-glucan fibres and on the other hand on the cooling rate kinetics in the production process.

The higher the content of β-glucan fibres in the solution, the more viscosity reducer (at least partially hydrolysed guar gum) must be introduced. In parallel, the slower the cooling kinetics in the production process, the less viscosity reducer (at least partially hydrolysed guar gum) must be introduced.

A method of determining the minimal content of viscosity reducer to be added is explained in example 2.

A method for preparing a thermized aqueous solution according to the invention includes a step of slow cooling of a thermized dispersion including:
water,
at least partially hydrolysed guar gum, and,
β-glucan fibres,
   under shearing, to a temperature between 4° C. and 30° C.

In particular, this method comprises the following successive steps:
a) Dispersing the at least partially hydrolysed guar gum and β-glucan fibres in water;
b) Heating the dispersion obtained following the preceding step to holding temperature and maintaining this dispersion at said holding temperature;
c) Slow cooling of the dispersion obtained following step b), under shearing, to a temperature between 4° C. and 30° C.

The inventors noted that, in addition to adding a viscosity reducer, the step of slow cooling under shearing was essential to introduce β-glucan fibres into an aqueous solution by avoiding both a too large increase in viscosity and the formation of a mass (gelling). Indeed, in the event of sudden cooling, gelling of the solution obtained is observed. This is also observed in the case of static cooling (i.e., without shearing).

Dispersions having viscosity values higher than 10,000 mPa s pose significant problems of pumpability. In the context of the present invention, a semi-fluid dispersion is a dispersion that has a viscosity (measured at 10° C.) lower than 10,000 mPa s.

Cooling is advantageously carried out at a maximum speed of 2° C./min.

For a fixed content of viscosity reducer (at least partially hydrolysed guar gum), the slower the cooling kinetics, the greater the content of β-glucan fibres that can be introduced.

From an economic point of view, it is difficult to conceive of a cooling period extending over more than one day. Cooling is thus advantageously carried out at a rate between 0.15° C./min and 1° C./min.

During cooling, the shear rate is generally between 10/sec and 800/sec, advantageously between 50/sec and 500/sec, more advantageously between 50/sec and 300/sec. It would seem that the shear rate has only a small impact on viscosity and gelling of the semi-fluid aqueous solution obtained. Shearing during cooling is, however, absolutely necessary.

Thermization conditions correspond to those typically used in the food industry. Thus, the holding temperature advantageously is between 80° C. and 95° C. In addition, the holding period advantageously varies from 2 minutes to 20 minutes.

The method can include, following step a) and before step b), a step in which fruit juice concentrate, concentrated fruit pulp, pieces of fruits and/or sugar are added to the dispersion obtained.

During step c), the aqueous solution is cooled to its usage or storage/preservation temperature. In the food industry, 10° C. is a typical preservation temperature.

According to another advantageous variant of the invention, β-glucan fibres are present in solid form in said food product. In particular, β-glucan fibres are present in the form of cereal flakes.

Typically, these products are in the form of dual-compartment units comprising a compartment containing the food product and another compartment containing the flakes rich in β-glucan fibres to be added in the food product before consumption.

In the context of the present invention, the food product is advantageously a semi-fluid food product chosen from the group comprised of soy-based products, fruit- and/or vegetable-based products, forages for cereal products, and dairy products. In particular, the food product is chosen from the group comprised of dairy products.

The dairy products are in particular fermented dairy products.

The term “fermented dairy products” means more particularly fermented dairy products ready for human consumption, i.e., fermented dairy foods. The present application more particularly relates to fermented milks and to yogurts. Said fermented dairy foods can alternatively be cottage cheese or “petits-suisses”.

“Fermented milks” and “yogurts” have the standard definitions used in the dairy industry, i.e., products which are intended for human consumption and which result from the acidifying lactic acid fermentation of a dairy substrate. These products may contain secondary ingredients such as fruits, plants, sugar, etc. Refer, for example, to French Decree no. 88-1203 of 30 Dec. 1988 relating to fermented milks and yogurts, published in the Official Journal of the French Republic on 31 Dec. 1988.

Reference can also be made to the “Codex Alimentarius” (prepared by the Codex Alimentarius Commission under the aegis of the FAO and the WHO, and published by the Information Division of the FAO, available online at http://www.codexalimentarius.net; see more particularly Volume 12 of the Codex Alimentarius “Codex standards for milk and milk products” and the standard “CODEX STAN A-11(a)-1975”).

The term “fermented milk” is thus reserved in the present application for dairy products prepared with a milk substrate which has undergone a treatment at least equivalent to pasteurisation, inoculated with microorganisms belonging to the species characteristic of each product. “Fermented milk” has not undergone any treatment to subtract a constitutive element of the milk substrate implemented and in particular has not undergone draining of the coagulum. Coagulation of “fermented milk” must not be obtained by means other than those resulting from the activity of the microorganisms used.

The term “yogurt” is reserved for fermented milk obtained, according to local and constant uses, by the development of specific thermophilic lactic acid bacteria named Lactobacillus bulgaricus and Streptococcus thermophilus, which must be found living in the finished product, at a concentration of at least 10 million bacteria per gram of the milk part.

In certain countries, regulations authorise the addition of other lactic acid bacteria in the production of yogurt, and notably the additional use of strains of Bifidobacterium and/or Lactobacillus acidophilus and/or Lactobacillus casei.

These additional lactic acid strains are intended to confer on the finished product various properties, such as to support the balance of intestinal flora or to modulate the immune system.

In practise, the expression “fermented milk” is thus generally used to indicate fermented milk other than yogurt and, depending on the country, it also can be called, for example, “Kefir”, “Kumiss”, “Lassi”, “Dahi”, “Leben”, “Filmjolk”, “Villi” or “Acidophilus milk”.

The quantity of free lactic acid contained in the fermented milk substrate should not be lower than 0.6 g per 100 g at the time of sale to the consumer, and the protein content provided by the milk part should not be lower than that of normal milk.

The name “cottage cheese” or “petit-suisse” is, in the present application, reserved for cheese which is not refined, not salted, and which has undergone fermentation by lactic acid bacteria only (and no fermentation other than lactic acid fermentation).

The dry matter content of cottage cheeses can be lowered to 15 g or 10 g per 100 g of cottage cheese, according to whether their fat content is 25% higher than 20 g, or at most equal to 20 g per 100 g of cottage cheese, after complete desiccation. The dry matter content of cottage cheese is between 13% and 20%. The dry matter content of petit-suisse is not lower than 23 g per 100 g of petit-suisse. It is generally between 25% and 30%. Cottage cheeses and petits-suisses are generally called “fresh” or “unripened” cheeses, used in a traditional way in the technical field of the present invention.

A further aim of the invention is a food product according to the invention as a functional food.

A functional food is a conventional food, or one which appears as such, which is part of a normal diet, and which has as a characteristic to provide beneficial physiological effects that exceed its usual nutritional functions or to reduce the risk of chronic diseases.

According to an advantageous variant of the invention, said functional food is intended to prevent diabetes, obesity and cardiovascular disease and to prevent and/or treat excess weight.

Indeed, it was noted that said functional food decreases postprandial insulinaemia while maintaining a normal glycaemic profile. This functional food can thus be used to slow the absorption of glucose by tissues, without causing hyperglycaemia which would be deleterious for the body.

This functional food can thus be useful in preventing type 2 diabetes. Indeed, type 2 diabetes is a pathology which occurs over a lifetime and which is caused by, amongst other things, overeating as well as the consumption of poor quality foods. The physiological changes which are associated with and which precede the disease are an increase in glucose intolerance, which is characterised by an imbalance between circulating glycaemia and insulin secretion. In other words, to maintain the same quantity of glucose in the blood, the glucose-intolerant subject must secrete a greater quantity of insulin. This phenomenon worsens during the genesis of the pathology, eventually arriving at the point at which the quantity of secreted insulin is extremely high but is no longer sufficient to re-establish glycaemia to its normal level, i.e., to basal glycaemia. This leads to two deleterious phenomena:
1) a too high concentration of blood glucose leading to a “poisoning” of the body which results in macro- and micro-vascular complications,
2) exhaustion of the pancreas which maintains hyperglycaemia by not secreting sufficient insulin.

Thus, a functional food that makes it possible to better control the ratio of the quantity of insulin secreted as a function of postprandial glycaemia can help prevent diabetes.

This functional food can also be useful in preventing obesity and excess weight via better management of the allocation of energy reserves over time.

Indeed, elevated insulin secretion during each postprandial phase, i.e., during almost all of the subject's waking hours, favours the use of glucose as an energy substrate at the expense of fatty acids. Fatty acids not used as energy substrates cause the subject's adipose reserves to grow, which then favours excess weight and, in the long term, the genesis of pathological obesity by a serious accumulation of weight.

Thus, a functional food that decreases insulin secretion enables better use of fatty acids and thus better regulation of fat mass in the long term, which is beneficial in preventing over weight and obesity.

This functional food can also be useful in preventing cardiovascular diseases via better control of postprandial insulinaemia and glycaemia.

Indeed, episodes of frequent hyperinsulinaemia and hyperglycaemia, as well as relative insensitivity to insulin, are risk factors of cardiovascular diseases. High insulin secretion is related to, amongst other things, an increase in hepatic secretion of C-reactive protein (CRP), which is an inflammatory agent generally used as a marker of atherothrombosis, which can cause a cardiac accident. Moreover, reducing episodes of hyperinsulinaemia and hyperglycaemia also decreases LDL-cholesterol and total cholesterol and improves the LDL/HDL cholesterol ratio. Both total cholesterol and LDL-cholesterol are recognised as precursors to heart disease.

Thus, a functional food that limits episodes of hyperinsulinaemia is beneficial in preventing cardiovascular diseases.

Finally, a further aim of the invention is the combination of guar gum and β-glucan fibres in the manufacture of a functional food intended to prevent diabetes, obesity and cardiovascular diseases and to prevent or treat excess weight.

In the functional food, the weight ratio of guar gum to beta-glucan fibres is between 2:1 and 4:1. In particular, the functional food includes 2% to 13% by weight guar gum and 2% to 9% by weight β-glucan fibres, compared to the total weight of said functional food. According to a preferred variant of the invention, the functional food is a dairy product.

The following examples illustrate the invention.

### DESCRIPTION OF THE FIGURES

FIG. 1: variation of postprandial glycaemia and insulinaemia as a function of time.

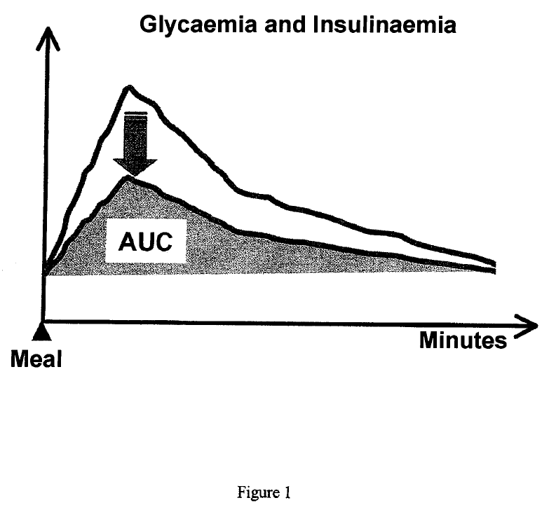

FIG. 2: variation of postprandial insulinaemia (pM) as a function of time (min) for the various products tested (cf. example 1).

FIG. 3: variation of postprandial glycaemia (mM) as a function of time (min) for the various products tested (cf. example 1).

FIG. 4: determination of the minimal content of PHGG, compared to the content of beta-glucan (% weight), for a cooling kinetics.

### EXAMPLE 1

### Effect of a Dairy Product Including Guar Gum and β-Glucan Fibres on Postprandial Insulinaemia in Healthy Subjects

The goal of this study is to determine the effect of a test fresh dairy product (FDP), as part of a meal, on postprandial insulinaemia in healthy subjects. This effect is compared with that of a control meal.

Materials and Methods

1. Subjects

Twelve subjects in good health, non-smokers, aged 18 to 45 years, were included in this study. In this study, to be certain to have enough subjects to evaluate, 12 healthy subjects were recruited and any subject who was eliminated early from the study was replaced.

->Inclusion criteria
1. Males and females aged 18 to 45 years;
2. Non-smokers;
3. Stable weight: 19-25 kg/m² BMI;
4. Healthy subjects with:
   Normal glucose tolerance;
   Normal blood profile for several metabolic health markers (complete blood count, gamma-GT, AST, ALT, glucose, TAGs, total cholesterol, HDL-cholesterol, LDL-cholesterol);
   Normal systolic blood pressure (100-150 mmHg);
   Normal diastolic blood pressure (60-90 mmHg);
   Normal resting heart rate (50-90 beats per minute after 3 minutes at rest).
5. Stable eating habits; normal amount of food; no history in terms of eating disorders or strict diet;
6. Moderate physical activity;
7. Able to go without food for at least 10 hours, the night before each test session;
8. Able to abstain from eating legumes and from drinking alcohol the day before each test session;
9. Able to complete 2 experimental sessions per week;
10. Subject covered by public or private medical insurance;
11. Attest to not be treated for anorexia, weight loss, or any form of treatment that can interfere with metabolism or dietary practises;
12. The subject has agreed in writing to take part in the study.

2. Food Products Tested

The test meals are composed of white bread, a fresh dairy product (FDP) and mineral water. In each meal, only the FDP is the differentiating element because it includes added active ingredients. These active ingredients are guar gum and oat beta-glucans.

Guar gum was made part of an FDP via a syrup. Beta-glucans are provided in the form of “flakes” contained in a “top cup” and mixed with the FDP at the moment of its consumption. The physical form of the introduction of beta-glucan into the product does not influence its effect.

The FDP tests containing the active ingredients and the recommended mixtures are as follows:

TABLE 1
Content of active ingredients in the various
dairy preparations tested

| Test FDP active | Oat beta- | |
|---|---|---|
| ingredient | glucan (g) | Guar gum (g) |
| Control meal | 0 | 0 |
| 1) β-glucan alone | 3.6 | 0 |
| 2) β-glucan + guar gum | 3.6 | 8.1 |
| 3) Guar gum alone | 0 | 8.1 |

Each FDP (control or test) is consumed with white bread and mineral water. Each meal provides a constant quantity of available carbohydrates of 50 g, a constant volume of water of 250 ml, and a constant quantity of FDP of 150 g. Available carbohydrates are provided by both the FDP and the bread. Thus, the macronutrient composition of the FDP and the bread makes it possible to determine the quantity of bread to consume with the FDP to provide 50 g of carbohydrates.

TABLE 2
Macronutrient composition for 100 g of the
products tested

| | Products tested | | | |
|---|---|---|---|---|
| | | | β- | |
| | | | glucan + | |
| | | | guar | Guar |
| Composition | Control | β-glucan | gum | gum |
| Moisture (g) | 88.1 | 76.5 | 70.9 | 81.1 |
| Protein (g) | 4.2 | 6.0 | 5.15 | 4.12 |
| Fat (g) | 0.1 | 1.3 | 1.06 | 0.12 |
| Total sugar (g) | 4.6 | 5.6 | 5.51 | 5.69 |
| Total starch (g) | 0 | 3.3 | 3.28 | 0 |
| Total carbohydrates | 4.6 | 8.9 | 8.8 | 5.7 |
| available in the | | | | |
| fresh dairy product | | | | |
| (g) | | | | |
| Dietary fibre (g) | 0 | 6.48 | 8.96 | 5.3 |
| White bread-qty. | 84.7 | 76.7 | 76.7 | 82.6 |
| consumed (g) | | | | |
| Quantity provided | 45.4 | 41.1 | 41.2 | 44.3 |
| (g) | | | | |
| Total carbohydrates | 50 | 50 | 50 | 50 |
| available in the | | | | |
| meal (g) | | | | |

3. Administration of the Products

Each of the 12 subjects taking part in the study consumes the 4 tested meals on only one occasion. At least one day separates the consecutive experimental sessions. Moreover, the subjects are asked to complete at least two experimental sessions per week during the major part of time that they take part in the study, but the minimum participation rate required is one experimental session per week. All the meals are given to the subjects in random order according to a randomization list drawn up by biostatisticians. The reference meal (standard dairy product+bread+water) and the tested meals (test dairy product+bread+water) are served to subjects in portions containing 50 grams of available carbohydrates. Each portion is weighed before and after consumption. The subjects consume each test or reference meal as well as water at a comfortable rhythm, but within 12 minutes maximum. All the tested meals are consumed by the subjects on an empty stomach in the morning, roughly at the same time that the subjects would normally consume their breakfast.

4. Experimental Method

4.1 Study Design and Experimental Protocol

This is a study of the short-term postprandial effects of the control product and of 3 products consumed during a meal on glycaemia and insulinaemia for a two-hour period. The results obtained with the products tested are compared with those obtained with the control meals (standard dairy product+bread+water). This is a crossover single-blind study.

4.2 General Experimental Conditions

The investigator first checks during a screening session that each subject is in good health (medical examination) and can take part in the study. The latter consists of repeated measurements of glycaemia and insulinaemia from blood samples taken from the fingertip. For each sample, the subjects place their hands in a bucket of hot water to increase blood circulation in their fingers. After 1-1.5 minutes, a blood sample is taken from their fingertip (−5 minutes), then another is taken five minutes later (0 minutes).

Each subject (seated at a table) is served a test meal or the control meal, which must be consumed within 12 minutes. A stop watch is started for each subject as soon as the subject starts to eat (0 minutes). Other blood samples are taken at 15, 30, 45, 60, 90 and 120 minutes after the start of the meal. During the 120 minutes of the experimental session, the subject remains seated in a quiet, stress-free environment.

4.3 Parameters measured

[symbol] Calculation of Areas Under the Curve for Insulinaemia and Glycaemia

For each 120-minute experimental session, plasma concentrations of glucose of the eight samples of plasma collected (2 in the so-called basal period, and 6 in the postprandial period) from the subject during this session are used to calculate the area under the curve (AUC) using the trapezoidal rule with the baseline truncated at zero. The baseline is defined herein as the mean between glucose concentrations at −5 minutes and 0 minutes. Any negative sector beneath the baseline is ignored. The AUC values allow comparison of the integrated effects of the products tested over a fixed period of time. An AUC value is calculated for each subject and for each product. The mean AUC for the 12 subjects is reported as a final AUC value of insulinaemia and glycaemia for each product.

The blood sample is collected in a 1.5 ml plastic microtube containing 10 international units of anticoagulant, heparin sodium salt. Just after the sample is taken, the blood is mixed with the anticoagulant by gently inverting the tube. The tube is then centrifuged. The plasma is then immediately transferred to a labelled plastic microtube and stored at −20° C. until analysed (<3 days for plasma glucose and <1 month for plasma insulin).

Plasma glucose concentrations are measured in duplicate from 5 μl samples using a spectrophotometer and glucose hexokinase/glucose-6-phosphate dehydrogenase enzymatic analysis. All eight blood samples collected from the same subject during an experimental session are analysed in the same series of analyses. Each series of analyses will be performed with standard controls and an internal serum control. Plasma insulin concentrations are measured by using a radioimmunology kit with tubes coated with antibody in solid phase.

Statistical Analyses

Statistical Objectives and Principles Used

The statistical analysis is performed by two complementary approaches:
Among the meals tested, which are those that significantly decrease, versus the control meals, the area under the curve of insulinaemia while maintaining normal glycaemia?
   Analysis of variance supplemented with tests comparing means are applied to respond to this approach.
Among the meals tested, which are those that significantly decrease the ratio between the insulinaemic response of the test meal and that of the control meal?
   The calculation of confidence in the relative response, resulting from the analysis of variance, is applied to respond to this approach.

Indicators

Indicators of the insulinaemic response are the area under the curve and the relative value of insulinaemia compared to the control meal. An ingredient is “active” when it significantly decreases the profile and/or the AUC.

Analysis of Deviations from the Protocol

Analysis of deviations from the protocol (minor and principal) is performed for each subject. Subjects presenting major deviations from the protocol are included in the ITT (intention to treat) population and are excluded from the PP (per protocol) population for the statistical analysis. Data is analysed for the following populations:
ITT population, i.e., all subjects enrolled in the study, randomised and receiving at least one of the products;
the PP population comprising the subjects included in the ITT population presenting no principal deviation from the protocol.
   Among the 12 Randomised Subjects None Presents Major Deviations from the Protocol.

Consolidation of the Data Matrix

As an introduction, the normality of the data is evaluated using the Shapiro test.

Treatment in Preparation of the Analysis of Variance

For any product taken individually, any subject having an insulinaemia and glycaemia value relative to the control product that is more than 2 standard deviations above or below the mean value of the product group will be removed to calculate the mean value of the group.

Descriptive Statistical Analysis

Descriptive statistics (mean, median, standard deviation, standard error of the mean (SEM), coefficient of variation (CV), minimum and maximum) are calculated for insulin and glucose plasma concentrations at each time point (−5, 0, 15, 30, 45, 60, 90 and 120 minutes) for each product tested and control product as well as for the insulinaemia and glycaemia values relative to the control for each product tested.

Analysis of Variance and Comparison of Means

Analysis of variance is performed to determine if there are significant differences between mean AUC values for insulinaemia and glycaemia following ingestion of the meals. If a produced effect is found to be statistically significant, a post-hoc test to compare means is performed (Dunnett's test) in order to identify the specific significant differences between the tested meals and the control meals.

Calculation of the Confidence Interval of the Relative Value

Analysis of the relative response of insulin and glycaemia compared to the control meal is performed by calculating the confidence interval resulting from the analysis of variance. If the confidence interval of the relative response for a given meal excludes the value 100, that indicates that it is different from the control meal.

Results

Study of insulinaemia

[symbol] Insulinaemic Profile and Area Under the Curve (AUC)

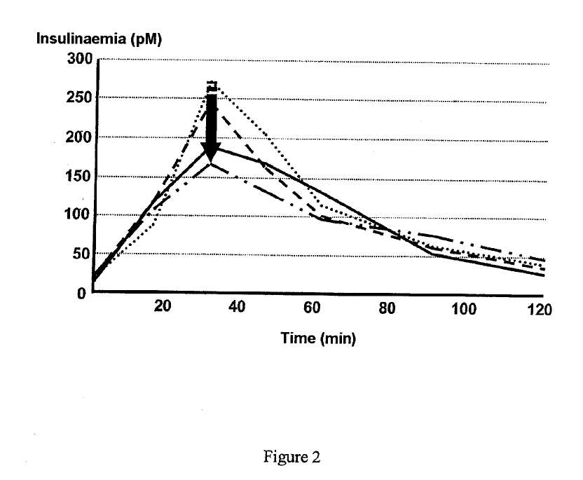

The results are presented in FIG. 2 which represent the evolution of the level of insulinaemia (pM) as a function of time (min)

Key:

...... control meals

------ guar gum only

—— beta-glucan only

— • • — beta-glucan+guar gum

Insulinaemia in the subjects before consumption of the meals is not significantly different from one meal to another (p=0.55); it is on the order of 21.5 μM.

In Comparison with Control Meals:
The meals containing the FDP with guar gum and beta-glucan (B-glucan) tend to decrease insulinaemic AUC compared to the control meal.
   The meal containing beta-glucans and guar gum tends to decrease AUC most markedly compared to the control meal.

[symbol] Insulinaemic Response (AUC)

The results of the analysis of variance of the insulinaemic response area under the curve are presented in following table 3:

TABLE 3
Summary of insulinaemia AUC

| | Product containing | Insulinaemia | |
|---|---|---|---|
| | for following | AUC | Pr > \|t\| |
| Meal | ingredient (s) | (pM/min) | (Dunnett's) |
| Control | FDP without active | 11812 | — |
| | ingredients | | |
| 1 | Beta-glucan | 12166 | 0.842 |
| 2 | Guar gum | 11102 | 0.886 |
| 3 | Beta-glucan + guar | 9390 | 0.037 |
| | gum | | |

Beta-glucan or guar gum alone does not significantly decrease insulinaemic response. The combination of these two ingredients in the same product allows a greater reduction in the area under the curve (−2422 pM/min; p<0.05) than the sum of the effects of the two ingredients taken separately (+356 pM/min and −710 pM/min, respectively). Thus, the reduction in the insulinaemic response of the meal including the FDP and the mixture (guar gum+beta-glucans) is on the order of 21%, whereas the reduction in the insulinaemic response of the meals including the FDP containing only guar gum or only beta-glucan is little changed (+3% and −6% for beta-glucan and guar gum, respectively) compared to the meal without the active ingredient.

The results of the insulinaemic response relative to the control meal are presented in table 4.

TABLE 4
Summary of relative responses of insulinaemia

| | | | | | | Reference |
|---|---|---|---|---|---|---|
| | | | | | | value |
| | Product | | | | | (100) |
| | containing the | Real | Estimated | | | included |
| | following | insulin | insulin | Negative | Positive | in the |
| Meal | ingredient(s): | response % | response % | limit | limit | interval |
| Control | FDP without | 100 | | | | |
| | active | | | | | |
| | ingredients | | | | | |
| 1 | β-glucan | 99.1 | 93.7 | 71.1 | 116.1 | Yes |
| 2 | Guar gum | 98.9 | 98.9 | 77.0 | 120.7 | Yes |
| 3 | B-glucan + guar | 78.9 | 74.2 | 51.6 | 96.8 | No |
| | gum | | | | | |

100 = value of the control meal
Confidence interval determined during the analysis of variance

This analysis makes it possible to show that the relative value of the insulinaemic response is significantly lower compared to the control meal, because the value 100 is excluded from the confidence interval of the meal containing beta-glucans and guar gum. This is not the case for the two meals containing one or other of the ingredients, whose values are not different from the control meal.

In conclusion, the beta-glucan+guar gum mixture significantly decreases (p<0.05) insulinaemic response by 21%, with a greater reduction compared to the two ingredients taken alone.

Study of glycaemia

[symbol] Glycaemic Profile and Area Under the Curve (AUC)

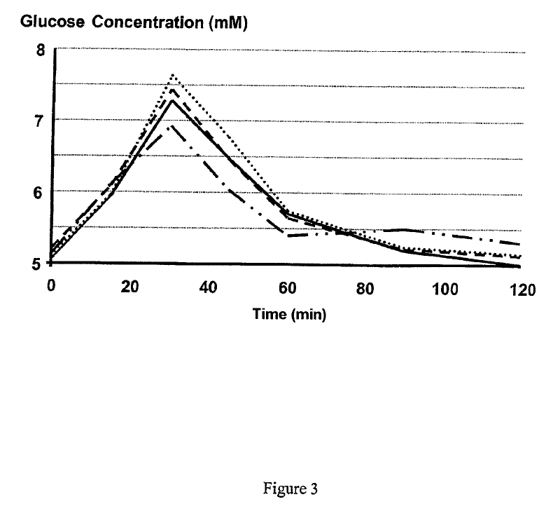

The results are presented in FIG. 3, which represents the evolution of glucose concentration (mM) as a function of time (min).

Key:

...... control meals

------ guar gum only

—— beta-glucan only

— • • — beta-glucan+guar gum

Glycaemia in the subjects before consumption of the meals is not significantly different from one meal to another (p=0.33); it is on the order of 5.15 mM.

The beta-glucans and guar gum added individually or in a mixture do not decrease the glycaemic response.

TABLE 5
Summary of the AUC of glycaemia

| | Product containing | | |
|---|---|---|---|
| | for following | Glycaemia | Pr > \|t\| |
| Meal | ingredient(s) | AUC (mM/min) | (Dunnett's) |
| Control | FDP without active | 101.6 | — |
| | ingredients | | |
| 1 | Beta-glucan | 95.3 | 0.803 |
| 2 | Guar gum | 89.4 | 0.630 |
| 3 | Beta-glucan + guar | 83.8 | 0.178 |
| | gum | | |

The beta-glucan+guar gum mixture maintains normal glycaemia.

The results of the relative glycaemic response compared to the control meal are presented in table 6.

TABLE 6
Summary of relative responses of glycaemia

| | | | | | | Reference |
|---|---|---|---|---|---|---|
| | | | | | | value |
| | Product | | | | | (100) |
| | containing the | Real | Estimeted | | | included |
| | following | glycaemia | glycaemia | Negative | Positive | in the |
| Meal | ingredient(s): | response % | response % | limit | limit | intervel |
| Control | FDP without | 100 | | | | |
| | active | | | | | |
| | ingredients | | | | | |
| 1 | β-glucan | 104.8 | 100.8 | 76.4 | 125.30 | Yes |
| 2 | Guar gum | 96.8 | 96.8 | 73.3 | 120.4 | Yes |
| 3 | B-glucan + guar | 88.3 | 84.6 | 60.2 | 109.1 | Yes |
| | gum | | | | | |

100 = value of the control meal
Confidence interval determined during the analysis of variance

The relative response of glycaemia compared to the control meal does not differ from one test meal to the other.

In conclusion, all the meals are equal with respect to glycaemic response.

Summary of Results

The table 7 below summarises the significance of the ingredients as well as the intensity of their impact on the drop in the various criteria studied versus the control meal over the course of the experiment.

TABLE 7
Effect of guar gum and beta-glucans on
insulinaemia and glycaemia

| Product | | | | |
|---|---|---|---|---|
| containing the | | Insulinaemia | | Glycaemia |
| following | Insulinaemia | relative to | Glycaemia | relative to |
| ingredient(s): | AUC | the control | AUC | the control |
| Beta-glucan | not | not | not | not |
| | significant | significant | significant | significant |
| Guar gum | not | not | not | not |
| | significant | significant | significant | significant |
| Beta-glucan + | ↓-p < 0.05 | ↓- | not | not |
| guar gum | | significantly | significant | significant |
| | | different | | |

General Conclusion

The implementation of a mixture of 3.6 g beta-glucan and 8.1 g guar gum in a fresh dairy product synergistically decreases insulinaemic response of the meal, while maintaining a peak and then normal postprandial glycaemic regression.

### Example 2

### Determination of Optimal Concentrations of Beta-Glucan and Partially Hydrolysed Guar Gum

A given quantity of partially hydrolysed guar gum (PHGG: SUNFIBER R®, Taiyo Kagaku, Fiderstadt, Germany) and beta-glucan fibres (BARLIV®, Cargill, Minneapolis, Minn., USA) is dispersed in water. This dispersion is then heated to 95° C. and then maintained at this temperature. The dispersion is finally slowly cooled (for 120 minutes), under shearing (150/sec), to 10° C.

The viscosity (measured using a PHYSICA UDS 200 rheometer, Anton Paar) of the solution obtained is measured just after manufacture (D0) and a day after its manufacture (D+1).

The appearance of the solution obtained is then evaluated at D+1. The comparison point for this evaluation is the appearance of a product not having undergone shearing but having undergone the same heat treatment.

The results obtained are given in table 8 below:

TABLE 8

| | | |
|---|---|---|
| | Viscosity (mPa · s | |
| Concentration (% w/w) | at 64/sec | Appearance |

| Beta-glucan | PHGG | D0 | D + 1 |
|---|---|---|---|
| 3 | 6 | 1870 | No gel |
| 3.5 | 5 | 2750 | Gel |
| 5 | 5 | 7520 | Gel |
| 5 | 7.5 | 5750 | Very slight |
| | | | gel |
| 8 | 15 | 7530 | No gel |

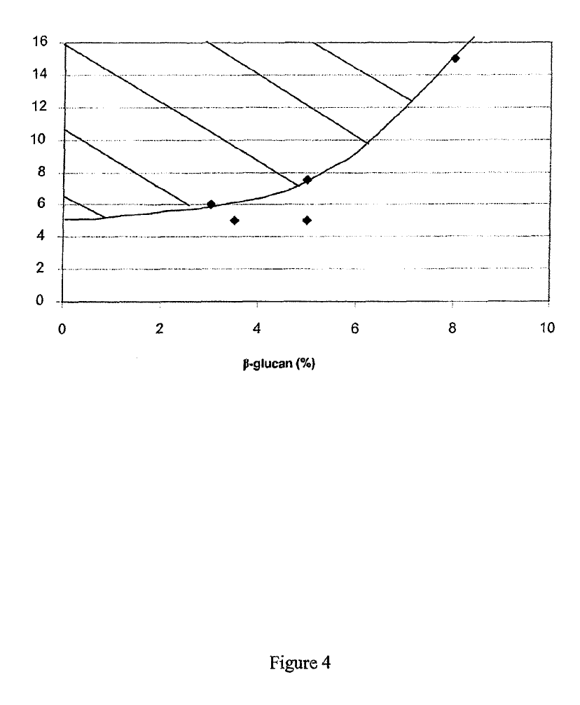

They are also presented in FIG. 4, in which PHGG content (in weight %) is plotted on the Y-axis and beta-glucan content (in weight %) is plotted on the X-axis.

The hatched zone, above the curve, corresponds to the area in which the dispersion does not gel (“no gel” area) whereas the area below the curve corresponds to a gelling area (“gel” area).

The curve obtained gives the minimal PHGG content necessary relative to the content of β-glucan desired, for a cooling kinetics.

The minimal contents of PHGG for other cooling kinetics can easily be determined by reproducing the protocol of example 2 adapted to the selected kinetics.

### Example 3

### Preparation of Fermented Dairy Products Containing Beta-Glucan

Stirred Yogurt Products:

A product close to a texturized stirred yogurt with fruits can be obtained by a 50/50 mixture of an unflavoured stirred yogurt (with a viscosity of 1050 mPa·s at 10° C.) and a 6.4% solution of β-glucan as described above.

The mixing operation does not present a particular difficulty and can be performed by using standard mixers.

This product has a viscosity of 1800 mPa s at 10° C., it has acceptable organoleptic properties and it is stable during preservation at 10° C. for 28 days.

125 g of this product contains 4 g of β-glucan.

Stirred Yogurt Products:

A product close to a more fluid stirred yogurt with fruits can be obtained by an 81/19 mixture of an unflavoured stirred yogurt (with a viscosity of 1050 mPa s at 10° C.) and a 6.4% solution of beta-glucan as described above.

The mixing operation does not present a particular difficulty and can be performed by using standard mixers.

This product has a viscosity of 1070 mPa s at 10° C., it has acceptable organoleptic properties and it is stable during preservation at 10° C. for 28 days.

125 g of this product contains 1.5 g of beta-glucan.

Fermented Milk Drinks:

A fermented milk beverage with fruits can be obtained by an 88% mixture of a fermented, unflavoured ready-to-drink milk (with a viscosity of 30 mPa s) and 12% of a fruit-juice-based preparation containing 6.4% of β-glucan as described above.

The mixing operation does not present a particular difficulty and can be performed by using standard mixers.

This product has a viscosity of 280 mPa·s at 10° C., it has acceptable organoleptic properties and it is stable during preservation at 10° C. for 28 days.

100 g of this beverage contains 0.75 g of β-glucan.

1. A semi-fluid food product including 2.5 g to 16 g of guar gum and 2.8 g to 11.3 g of beta-glucan fibres, per portion of said food product, said portions being between 125 g and 250 g, wherein the weight ratio of guar gum to beta-glucan fibres is between 2:1 and 4:1.

2. The food product according to claim 1, wherein it includes 8 g to 12 g of guar gum and 3 g to 4 g of beta-glucan fibres, per portion of said food product, said portions being between 125 g and 250 g.

3. The food product according claim 1, wherein the beta-glucan fibres are dispersed in said food product.

4. The food product according to claim 1, wherein the beta-glucan fibres are present in a solid form in said food product.

5. The food product according claim 1, wherein the source of the beta-glucan fibres is a cereal extract chosen from the group comprised of barley and oats, in particular oats.

6. The food product according claim 1, wherein it is chosen from the group comprised of soy-based products, fruit- and/or vegetable-based products, forages for cereal products, and dairy products.

7. The food product according to claim 6, wherein it is chosen from the group comprised of dairy products.

8. (canceled)

9. A method for preventing diabetes, obesity and cardiovascular diseases and for preventing and/or treating overweight comprising the administration to a patient in need thereof of an effective amount of the food product according to claim 1.

10. A method for decreasing postprandial insulinaemia while maintaining a normal glycaemic profile comprising the administration to a patient in need thereof of an effective amount of the food product according to claim 1.

11. A method for slowing the absorption of glucose comprising the administration to a patient in need thereof of an effective amount of the food product according to claim 1.

12. A method for reducing blood cholesterol level comprising the administration to a patient in need thereof of an effective amount of the food product according to claim 1.

13. A method for preventing diabetes, obesity and cardiovascular diseases and for preventing or treating overweight comprising the administration to a patient in need thereof of an effective amount of a functional food containing a combination of guar gum and β-glucan fibres with the weight ratio of guar gum to beta-glucan fibres is between 2:1 and 4:1.

14. The method according to claim 13, wherein the functional food is a dairy product.

15. The food product according to claim 5 wherein the beta-glucan fibres is an oat extract.
